(19) United States
(12) Patent Application Publication
(10) Pub. No.: US 2022/0378599 A1
(43) Pub. Date: Dec. 1, 2022

(54) ASSISTIVE DEVICE STRUCTURE FOR POSITIONING AND PRESSURE RELIEF

(71) Applicants: SY-WEN HORNG, Taipei City (TW); LONG-YING CHENG, Taipei City (TW); CHI-WEI HUNG, Taipei City (TW); HSIANG-JUNG HUNG, Taipei City (TW); LI-CHE HUNG, Taipei City (TW)

(72) Inventors: SY-WEN HORNG, Taipei City (TW); LONG-YING CHENG, Taipei City (TW); CHI-WEI HUNG, Taipei City (TW); HSIANG-JUNG HUNG, Taipei City (TW); LI-CHE HUNG, Taipei City (TW)

(21) Appl. No.: 17/663,948

(22) Filed: May 18, 2022

(30) Foreign Application Priority Data
May 25, 2021 (TW) 110118901

(51) Int. Cl. A61F 5/30

(52) U.S. Cl. CPC A61F 5/30; A61H 1/008

(57) ABSTRACT

An assistive device structure for positioning and pressure relief is provided, including a first elastic layer and a second elastic layer, which are attached by using a high-frequency encapsulation process, sealing, bagging, thermoforming, or an integrally molding process. Each of the first and second elastic layers has a bottom surface and an arc surface disposed opposite to each other. The arc surface includes two protrusions and a recess formed there in between. The two protrusions have different heights. A hollow area is disposed in the recess of the first and second elastic layers. Based on such structure, the bottom surfaces of the first and second elastic layers are attached to form the proposed assistive device structure for a user to lean against and providing multiple positioning effects and pressure relief More than four axial directions of supporting forces are generated to effectively enhance muscle relaxation and stress relief.

This application claims priority of Application No. 110118901 filed in Taiwan on 25 May 2021 under 35 U.S.C. § 119; the entire contents of all of which are hereby incorporated by reference.

### BACKGROUND OF THE INVENTION

### Field of the Invention

The present invention relates to a certain type of aid which is applicable to support positioning and adjust the gesture of a user. More particularly, it is related to an assistive device structure for positioning and pressure relieving, which also provides a variety of lying surfaces for people to use and effectively promotes neuromuscular relaxation and stress relief thereof.

### Description of the Prior Art

In recent years, people's lifestyles have become more and more rushed and tense along with the rapid developments of information science and evolutions of current technologies. If one's pressure can not be adjusted and released properly, it affects people's health not only physically but also mentally. Currently, there are several existing health care equipment on the market for consumers to purchase and for pressure relief to use at home, such as massage chairs, fascia guns, etc. However, since these products are usually high priced and difficult to afford, they still can not be widely used by the general public.

For solving the issues, after years of research and clinical testing, the academic and related industries have developed a variety of manual therapy supplementary theories, including Craniosacral Therapy (CST), cranial nerve relaxation, Chiropractic, soft tissue chiropractic, osteopathic, and so on. Among them, the feature of CST lies in: a practitioner can simply press the subject with a gentle force of less than five grams through his finger to help the subject increase balance of its body movements and normal flow of its body fluids. For example, the head and dura mater will follow circulation of the cerebrospinal fluid and adjust the hydraulic pressure of the cerebrospinal fluid for enhancing health of the subject. Although these manual therapy supplementary medications provide gentle and suitable methodologies for the general public to learn and use, they have not been recognized by the general mainstream medication group until now. The reason is that these manual treatments still lack rigorous scientific evidence for effective verification. In addition, these manual treatments are very likely to be affected by personal factors, including different practitioners or practitioners in different situations and occasions. Therefore, it is believed that there still exists a great amount of variability in these manual treatments.

And furthermore, in addition to the professional assistive device structures used by the health care specialists, a plurality of assistive device structures may further include: home life aids, personal mobility aids, personal medical aids, personal care and protection aids, orthoses and prosthetics, visually impaired aids, and hearing aids, etc. In general, a user can improve, monitor, or maintain personal medical status with the aids, and can also use the aids to improve body weight distribution on his own, in order to maintain an upright position and reduce occurrences of pressure ulcers and to relax cramp muscles. Healthy people may also adjust their postures by using positioning aids. For instance, when the user is lying down and in sleep, the positioning aid can be placed on his caudal vertebra for reducing the pressure on the lumbar vertebrae. Alternatively, if the user is lying on his side, it is also feasible to use the positioning aid to separate his legs, so that the legs extend parallel from his hip joint, and therefore prevent torsion of the hip joint effectively. However, since these currently used positioning aids are merely simple pillows or cushions made of cotton, linen or synthetic fibers, their positioning styles and patterns are limited while in use. Moreover, it is not possible to combine these positioning aids with the aforementioned manual therapies, such as craniosacral therapy (CST) effectively. Therefore, on account of above, to overcome the abovementioned problems, it should be obvious that there is indeed an urgent need for the professionals in the field for a novel and improved assistive device structure to be developed that can effectively solve the above mentioned issues occurring in the prior design. By using such an improved and modified assistive device structure, it is effective and beneficial for user's positioning and pressure relieving. Also, optimal results for multi-axial support forces can be generated and provided through the present invention. The specific implementations of the present invention will be fully introduced and described in the following paragraphs.

### SUMMARY OF THE INVENTION

In order to overcome the abovementioned disadvantages, one major objective in accordance with the present invention is to provide a novel assistive device structure for positioning and pressure relief By employing the proposed assistive device structure for positioning and pressure relief of the present invention, it can effectively adjust a user's position and support where the user is leaning against. Besides, a variety of leaning surfaces of the proposed device structure can be selected by the user according to different use requirements and conditions, such that the user is provided with a much more comfortable experiencing feeling.

And yet, another major objective in accordance with the present invention is provided for the novel assistive device structure for positioning and pressure relief. When the assistive device structure is applied to support and protect the user's spine, the present invention does not need to use the instantaneous bursting force like it was in the conventional chiropractic therapy. Instead, a relatively gentle, safe, and non-destructive supporting force can be provided by using the proposed device structure of the present invention, in which a longer period of soothing extension is employed.

And also, another further major objective in accordance with the present invention is provided for the disclosed assistive device structure for positioning and pressure relief. It fully demonstrates and verifies that the dialectical relationship between soft tissue adjustment and vertebral restoration is applicable and mutually dependent. The assistive device structure for positioning and pressure relief disclosed in the present invention not only shows significant effects on most of the rachiopathy caused by the abnormal structure of the spine. When it is applied to scoliosis, it can also effectively provide decompression and pressure relief to support and protect the spine. In addition, effective traction forces and reconciling the spine vertically are accomplished by using the present invention.

Therefore, for achieving the above-mentioned objectives, the technical solutions of the present invention are aimed to provide an assistive device structure for positioning and pressure relief, which comprises a first elastic layer and a second elastic layer. The first elastic layer includes a first bottom surface and a first arc surface. The first bottom surface and the first arc surface are disposed opposite to each other, and the first arc surface includes two first protrusions and a first recess which is formed between the two first protrusions. The two first protrusions have different heights. The second elastic layer includes a second bottom surface and a second arc surface, wherein the second bottom surface and the second arc surface are disposed opposite to each other. The second arc surface includes two second protrusions and a second recess which is formed between the two second protrusions. The two second protrusions have different heights. According to the present invention, the first bottom surface of the first elastic layer and the second bottom surface of the second elastic layer are attached, so as to form the disclosed assistive device structure for positioning and pressure relief for a user to lean against.

In one embodiment of the present invention, the first elastic layer and the second elastic layer can be attached to each other by using a high-frequency encapsulation process, a sealing process, a bagging process, or a thermoforming process.

In another embodiment of the present invention, the first elastic layer and the second elastic layer can be attached to each other by using an integrally molding process.

According to one embodiment of the present invention, when the first elastic layer and the second elastic layer are attached to each other, one of the two first protrusions with a higher height in the first elastic layer and one of the two second protrusions with a higher height in the second elastic layer are commonly disposed in a same axial direction, and another of the two first protrusions with a lower height in the first elastic layer and another of the two second protrusions with a lower height in the second elastic layer are commonly disposed in another axial direction.

According to another embodiment of the present invention, when the first elastic layer and the second elastic layer are attached to each other, one of the two first protrusions with a higher height in the first elastic layer and one of the two second protrusions with a lower height in the second elastic layer are commonly disposed in a same axial direction, and another of the two first protrusions with a lower height in the first elastic layer and another of the two second protrusions with a higher height in the second elastic layer are commonly disposed in another axial direction. By adopting such design manners, the assistive device structure disclosed by the present invention is able to provide a plurality of various positioning styles for the user to lean against, and multi-axial supporting forces are generated.

According to the embodiment of the present invention, the first elastic layer can be composed of a plurality of coiled elastic threads, and the elastic threads are made of plastic materials (for example: TPU, POE, PE, EVA, PP, PVC, PET or a mixture of the aforementioned plastic materials).

The first elastic layer provides a first arc surface hardness in the region which is adjacent to the first arc surface. The first elastic layer provides a first bottom surface hardness in the region which is adjacent to the first bottom surface. The region between the first arc surface and the first bottom surface of the first elastic layer is a first intermediate layer, and the first elastic layer provides a first intermediate layer hardness in the first intermediate layer. A spacing between the elastic threads disposed adjacent to the first arc surface of the first elastic layer is less than a spacing between the elastic threads disposed adjacent to the first bottom surface of the first elastic layer, and the spacing between the elastic threads disposed adjacent to the first bottom surface of the first elastic layer is less than a spacing between the elastic threads disposed in the first intermediate layer of the first elastic layer, such that the first arc surface hardness is greater than the first bottom surface hardness, and the first bottom surface hardness is greater than the first intermediate layer hardness.

Similarly, the second elastic layer can be composed of a plurality of coiled elastic threads, and the elastic threads can be made of plastic materials (for example: TPU, POE, PE, EVA, PP, PVC, PET or a mixture of the aforementioned plastic materials). The second elastic layer provides a second arc surface hardness in the region which is adjacent to the second arc surface. The second elastic layer provides a second bottom surface hardness in the region which is adjacent to the second bottom surface. The region between the second arc surface and the second bottom surface of the second elastic layer is a second intermediate layer, and the second elastic layer provides a second intermediate layer hardness in the second intermediate layer. A spacing between the elastic threads disposed adjacent to the second arc surface of the second elastic layer is less than a spacing between the elastic threads disposed adjacent to the second bottom surface of the second elastic layer, and the spacing between the elastic threads disposed adjacent to the second bottom surface of the second elastic layer is less than a spacing between the elastic threads disposed in the second intermediate layer of the second elastic layer, such that the second arc surface hardness is greater than the second bottom surface hardness, and the second bottom surface hardness is greater than the second intermediate layer hardness.

According to the embodiment of the present invention, the first arc surface hardness, the first bottom surface hardness, the first intermediate layer hardness of the first elastic layer, and the second arc surface hardness, the second bottom surface hardness, and the second intermediate layer hardness of the second elastic layer are different. Besides, the coiled elastic threads are also connected vertically to each other, and therefore providing longitudinal supporting forces. As such, by adjusting distribution densities of the elastic threads in the first elastic layer and in the second elastic layer, the formed assistive device structure by the present invention is able to provide a variety (at least six different combination) of hardness, including the first arc surface hardness, the first bottom surface hardness, the first intermediate layer hardness, the second arc surface hardness, the second bottom surface hardness, and the second intermediate layer hardness. And based on such technical features, the user is provided with a variety of positioning experiences and supporting forces.

Furthermore, according to the assistive device structure for positioning and pressure relief of the present invention, a hollow area can be further disposed in either the first recess or the second recess to reduce force and pressure concentration when the user is leaning against it. In addition, each first protrusion and second protrusion disposed on opposite sides of the hollow area includes a supporting portion, respectively. A peripheral portion of the first elastic layer and the second elastic layer is configured between the adjacent supporting portions. According to the designed structure, when the user is leaning against, the supporting portion disposed on one side of the hollow area is used to support occipital bone and cervical spine of the user, while the supporting portion disposed on opposite side of the hollow area is used to support parietal bone of the user. The hollow area is used to accommodate posterior pole of occipital bone of the user, such that various supporting heights and supporting hardness can be provided due to the different heights of the two first protrusions in the first elastic layer and of the two second protrusions in the second elastic layer.

In one embodiment, a width of each supporting portion is greater than its length thereof.

In one embodiment, an edge of the supporting portion adjacent to the hollow area is an arc.

According to the embodiment of the present invention, a plurality of cutting lines are formed along the hollow area in an extending direction of the edge of the supporting portion. In one embodiment, each of the cutting lines is designed to have 2 to 4 cm in length. Each of the cutting lines goes deep into a supporting portion, such that the supporting portion is movable relative to the peripheral portion. Due to such design manners, when the user is leaning against, a height of the peripheral portion is higher than that of the supporting portion, so as to prevent the user's head from moving and avoid a crick in the user's neck effectively.

In another aspect, regarding the patterns of the cutting lines, the plurality of cutting lines may also be alternatively configured in the first recess or in the second recess, and the plurality of cutting lines are formed in a symmetrical pattern, such as H-shaped, I-shaped, O-shaped, X-shaped, or the figure-eight “8”-shaped.

And moreover, in order to meet the practical application requirements, the assistive device structure for positioning and pressure relief disclosed by the present invention can be further applied to sleep detection. In such an embodiment, a sensor can be disposed and embedded in the first elastic layer or in the second elastic layer to detect and measure the user's sleep or leaning, using, and other conditions.

And yet, in another aspect, according to the embodiment of the present invention, when the first elastic layer and the second elastic layer are attached to each other, one first protrusion with a higher height in the first elastic layer and one second protrusion with a lower height in the second elastic layer are commonly disposed in a same axial direction, and another first protrusions with a lower height in the first elastic layer and another second protrusion with a higher height in the second elastic layer are commonly disposed in another axial direction, under such a condition, a gap will be formed between the edge of the supporting portion in the first elastic layer and the edge of the supporting portion in the second elastic layer. Due to the gap, the supporting portion which supports the user's occipital bone, can be prevented from over bending or deformation when the user is leaning against the disclosed assistive device structure.

These and other objectives of the present invention will become obvious to those of ordinary skill in the art after reading the following detailed description of preferred embodiments.

It is to be understood that both the foregoing general description and the following detailed description are exemplary and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings:

FIG. 1 illustratively shows a schematic view of a first elastic layer of the disclosed assistive device structure for positioning and pressure relief in accordance with one embodiment of the present invention.

FIG. 2 illustratively shows a schematic view of a second elastic layer of the disclosed assistive device structure for positioning and pressure relief in accordance with one embodiment of the present invention.

FIG. 3 illustratively shows a schematic diagram when the first elastic layer and the second elastic layer are attached to form the disclosed assistive device structure according to one embodiment of the present invention.

FIG. 4 illustratively shows a schematic diagram when the first elastic layer and the second elastic layer are attached to form the disclosed assistive device structure according to another embodiment of the present invention.

FIG. 5 shows a schematic diagram of distribution density of the first elastic layer according to FIG. 1 of the present invention.

FIG. 6 shows a schematic diagram of distribution density of the second elastic layer according to FIG. 2 of the present invention.

FIG. 7 illustratively shows a schematic diagram of the disclosed assistive device structure when a hollow area is further disposed.

FIG. 8 shows an upper view of FIG. 7.

FIG. 9 shows a schematic structure diagram of a human skull.

FIG. 10 illustratively shows a schematic diagram of the disclosed assistive device structure when the supporting portions are movable relative to the peripheral portion.

FIG. 11A illustratively shows a schematic diagram of the disclosed assistive device structure when the H-shaped cutting line is formed therein.

FIG. 11B illustratively shows a schematic diagram of the disclosed assistive device structure when the I-shaped cutting line is formed therein.

FIG. 11C illustratively shows a schematic diagram of the disclosed assistive device structure when the O-shaped cutting line is formed therein.

FIG. 11D illustratively shows a schematic diagram of the disclosed assistive device structure when the X-shaped cutting line is formed therein.

FIG. 11E illustratively shows a schematic diagram of the disclosed assistive device structure when the “8”-shaped cutting line is formed therein.

FIG. 12 illustratively shows a schematic diagram of the disclosed assistive device structure when a gap is formed between the edge of the supporting portion in the first elastic layer and the edge of the supporting portion in the second elastic layer.

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

The embodiments described below are illustrated to demonstrate the technical contents and characteristics of the present invention and to enable the persons skilled in the art to understand, make, and use the present invention. However, it shall be noticed that, it is not intended to limit the scope of the present invention. Therefore, any equivalent modification or variation according to the spirit of the present invention is to be also included within the scope of the present invention.

In view of the foregoing deficiencies as discussed in the aforementioned prior art, the present invention is aimed to provide a modified assistive device structure. By employing the assistive device structure disclosed in the present invention, it achieves to provide pressure-releasing and positioning effect. Furthermore, a user is allowed to select and use different surfaces of the assistive device structure according to its practical requirements and using conditions, so as to have an optimized supporting effect and comfortable experience.

It is known that spine stands for a second lifeline of a human body, occupies the central part in the human body and mainly dominates the human health. If the spine compresses nerves in the human body, the human's brain cannot function accurately and thus functional disorders occur, thereby affecting health of the human body severely. For health recovery, “Spinal Manipulation Made Simple, A Manual of Soft Tissue Techniques” also known as Chiropractic, has been proposed to focus on balance of a human body to discover its interior mysteries. In general, Chiropractic uses a single burst of pressure to push the spine, that is malpositioned, back into its normal position. However, the burst is always not comfortable, and to avoid such uncomfortableness, the present invention is aimed to provide an improved assistive device structure for positioning and pressure relief By adopting the present invention, it is able to provide a variety of adjustment positions, including height, hardness, supporting effect, angle, and arc, etc., and also make the user's lying portion obtain non-linear arc stretching, traction, and relaxation. The assistive device structure disclosed in the present invention for positioning and pressure relief, can be applied to stretch not only spine, but also cervical vertebrae, thoracic vertebrae, lumbar vertebrae, and so on. In addition, according to different bending moments, the disclosed assistive device structure is able to generate different acting or pulling forces as well.

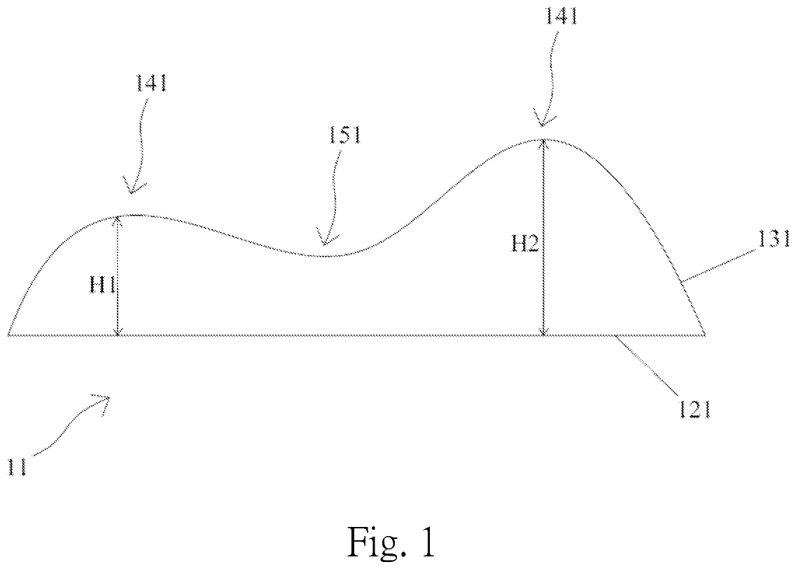

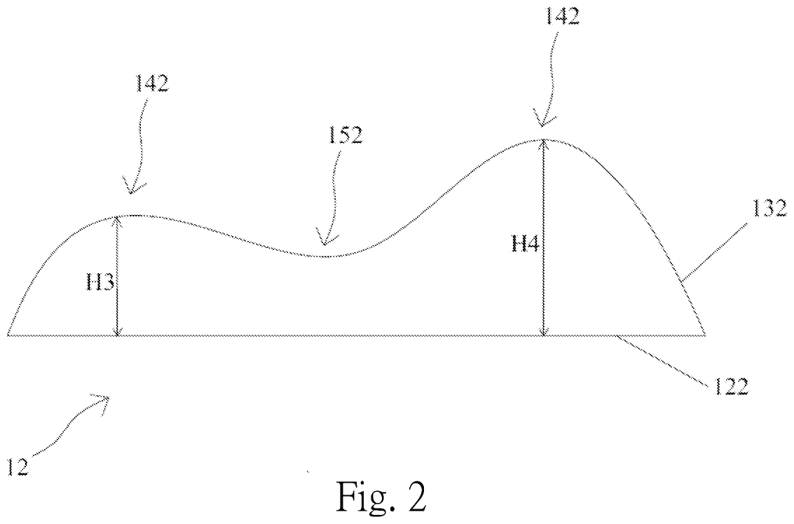

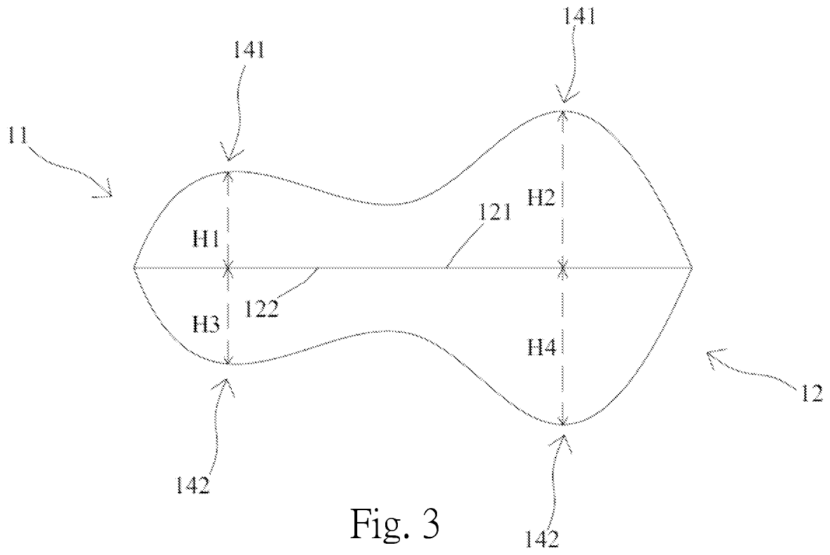

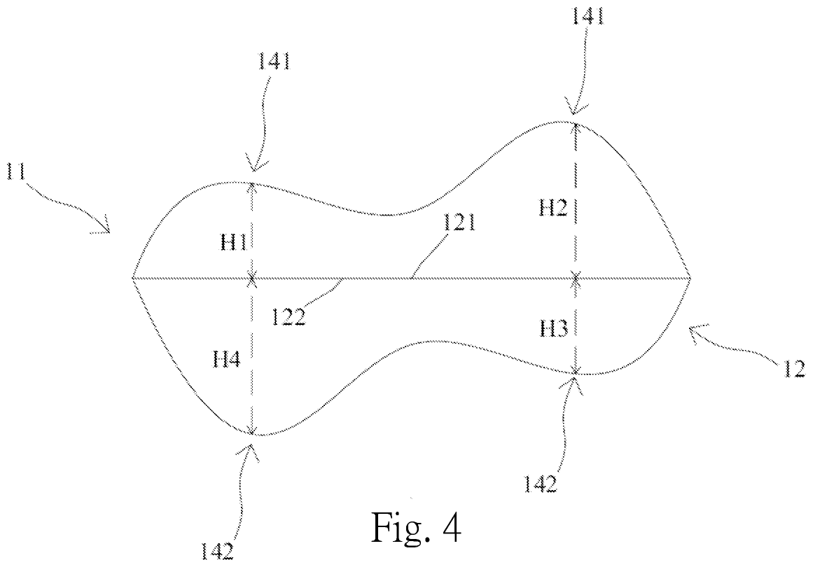

First of all, please refer to FIG. 1, which illustratively shows a schematic view of a first elastic layer of the disclosed assistive device structure for positioning and pressure relief in accordance with one embodiment of the present invention. As shown, the first elastic layer 11 includes a first bottom surface 121 and a first arc surface 131. The first bottom surface 121 and the first arc surface 131 are opposite. Specifically, the first arc surface 131 includes two first protrusions 141 and a first recess 151 which is formed between the two first protrusions 141. The two first protrusions 141 have different heights H1, H2. FIG. 2 illustratively shows a schematic view of a second elastic layer of the disclosed assistive device structure for positioning and pressure relief in accordance with the embodiment of the present invention. As shown, the second elastic layer 12 includes a second bottom surface 122 and a second arc surface 132. The second bottom surface 122 and the second arc surface 132 are opposite. Specifically, the second arc surface 132 includes two second protrusions 142 and a second recess 152 which is formed between the two second protrusions 142. The two second protrusions 142 have different heights H3, H4. According to the present invention, the first bottom surface 121 of the first elastic layer 11 shown in FIG. 1 is attached to the second bottom surface 122 of the second elastic layer 12 shown in FIG. 2, so as to form the disclosed assistive device structure for positioning and pressure relief for a user to lean against. The schematic diagram of the structure after the first and second elastic layers are attached is provided as shown in FIG. 3 and FIG. 4 of the present invention.

Please refer to FIG. 3 for the schematic diagram when the first elastic layer 11 and the second elastic layer 12 are attached, in which it shows that a first protrusion 141 with a higher height H2 in the first elastic layer 11 and a second protrusion 142 with a higher height H4 in the second elastic layer 12 are commonly disposed in a same axial direction. And, a first protrusion 141 with a lower height H1 in the first elastic layer 11 and a second protrusion 142 with a lower height H3 in the second elastic layer 12 are commonly disposed in another axial direction. As a result, when the user is leaning against the structure, it is optional to place the parietal bone of its head on the first protrusion 141 of the first elastic layer 11 having a higher height H2, or on the first protrusion 141 of the first elastic layer 11 having a lower height H1. Alternatively, if the structure in FIG. 3 is reversed (upside down, indicating that second elastic layer 12 is facing upward, and the first elastic layer 11 is facing downward), and the user is also able to optionally place the parietal bone of its head on the second protrusion 142 of the second elastic layer 12 having a higher height H4, or on the second protrusion 142 of the second elastic layer 12 having a lower height H3. By such design manners, the disclosed assistive device structure for positioning and pressure relief in the present invention provides at least four different positions for the user to lean against, thereby offering a variety of supporting forces for effective positioning and pressure relieving.

Similarly, please refer to FIG. 4, which shows a variant embodiment of the invention. As shown in FIG. 4, when the first elastic layer 11 and the second elastic layer 12 are attached, it is also applicable that a first protrusion 141 with a higher height H2 in the first elastic layer 11 and a second protrusion 142 with a lower height H3 in the second elastic layer 12 are commonly disposed in a same axial direction. And, a first protrusion 141 with a lower height H1 in the first elastic layer 11 and a second protrusion 142 with a higher height H4 in the second elastic layer 12 are commonly disposed in another axial direction. As a result, when the user is leaning against the structure, it is optional to place the parietal bone of its head on the first protrusion 141 of the first elastic layer 11 having a higher height H2, or on the first protrusion 141 of the first elastic layer 11 having a lower height H1. Alternatively, if the structure in FIG. 4 is reversed (upside down, indicating that the second elastic layer 12 is facing upward, and the first elastic layer 11 is facing downward), the user is also able to optionally place the parietal bone of its head on the second protrusion 142 of the second elastic layer 12 having a higher height H4, or on the second protrusion 142 of the second elastic layer 12 having a lower height H3. As a result, in view of the novel design manners as mentioned in the present invention, the disclosed assistive device structure for positioning and pressure relief of the invention further provides another four different positions for the user to lean against, thereby offering a much more variety of supporting forces for effective positioning and pressure relieving.

Furthermore, in order to provide an even more better supporting effect, the first elastic layer 11 or the second elastic layer 12 of the present invention can also be designed to have different hardness depending on actual product requirements. In either the embodiment as shown in FIG. 3 or the embodiment as shown in FIG. 4, the first elastic layer 11 and the second elastic layer 12 of the present invention can be attached to each other by using a high-frequency encapsulation process, a sealing process, a bagging process, or a thermoforming process. Even more, through a bundling or butt joint process, the two corresponding bottom surfaces of the first elastic layer 11 and the second elastic layer 12 (the first bottom surface 121 and the second bottom surface 122) can be attached as well. Overall, for people who have ordinary knowledge and are skilled in the art, it is allowed to make modifications regarding methodologies to attach the elastic layers according to their actual design requirements and implementation levels without departing from the technical spirits of the present invention. It is assured that these modifications still fall into the claim scope of the present invention, and the present invention is not limited to the abovementioned joint means. For instance, according to other embodiment of the present invention, the first elastic layer 11 and the second elastic layer 12 may also be attached to each other by using an integrally molding process so as to form the assistive device structure for a user to lean against and for positioning and pressure relief of the invention. Such an integrally molding process can also be used to achieve the purpose of the present invention.

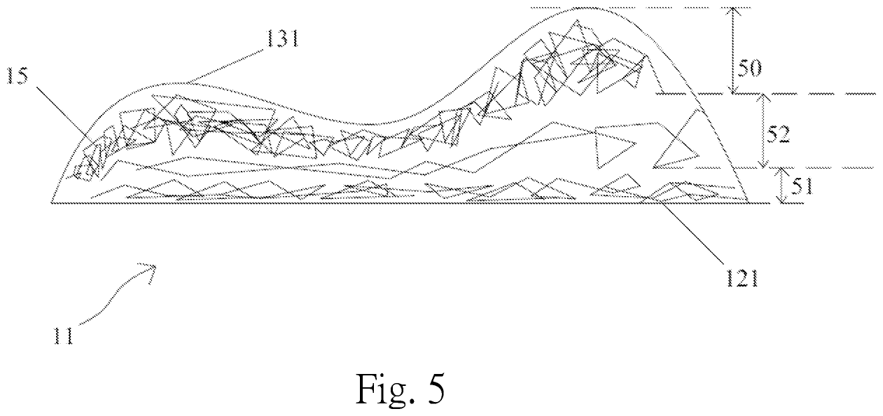

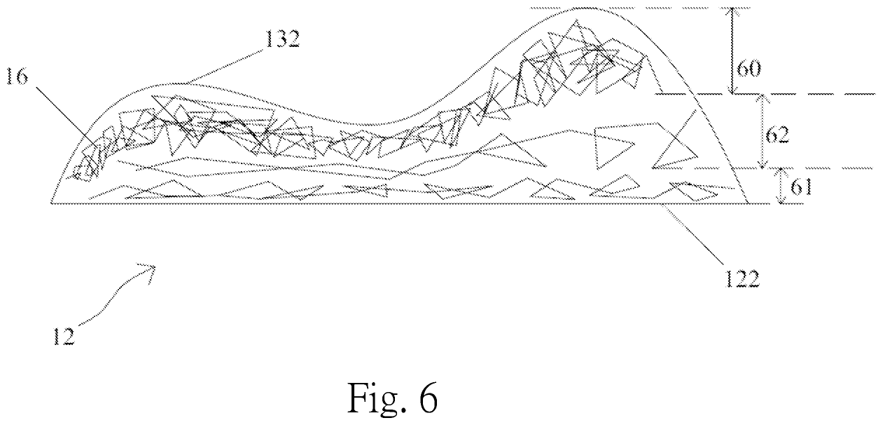

In the following, the Applicant further provides relevant descriptions regarding the composition materials, the softness and the hardness of the first elastic layer 11 and the second elastic layer 12 disclosed in the present invention. Please refer to the structures, components, and element symbols as shown in FIG. 5 and FIG. 6 at the same time. The present invention hereby provides a detailed description as follows. Firstly, as shown in the embodiment illustrated in FIG. 5, the first elastic layer 11 can be composed of a plurality of coiled elastic threads 15, and the elastic threads 15 are flexible and have elasticity. The elastic threads 15 are made of plastic materials, such as TPU (Thermoplastic polyurethane), POE (Polyolefin Elastomer), PE (polyethylene), EVA (Ethylene vinyl acetate), PP (Polypropylene), PVC (Polyvinylchlorid), PET (polyethylene terephthalate) and other plastic materials, or a mixture of the aforementioned plastic materials. In general, for different design requirements, it is feasible to choose the elastic threads 15 with different thread diameters, to adjust its original spacing or curling between the elastic threads 15, or to use different materials and/or thicknesses of the elastic threads 15, etc., such that the elastic threads 15 can have different densities, and thus provide different supporting effects and hardness for the first elastic layer 11. In one embodiment of the present invention, for example, the first elastic layer 11 provides a first arc surface hardness in the region 50 which is adjacent to the first arc surface 131. The first elastic layer 11 provides a first bottom surface hardness in the region 51 which is adjacent to the first bottom surface 121. The region between the first arc surface 131 and the first bottom surface 121 of the first elastic layer 11 is a first intermediate layer 52, and the first elastic layer 11 provides a first intermediate layer hardness in the first intermediate layer 52. When a spacing between the elastic threads 15 disposed adjacent to the first arc surface 131 of the first elastic layer 11 is less than a spacing between the elastic threads 15 disposed adjacent to the first bottom surface 121 of the first elastic layer 11, and the spacing between the elastic threads 15 disposed adjacent to the first bottom surface 121 of the first elastic layer 11 is less than a spacing between the elastic threads 15 disposed in the first intermediate layer 52 of the first elastic layer 11, the present invention achieves in controlling the first arc surface hardness greater than the first bottom surface hardness, and the first bottom surface hardness greater than the first intermediate layer hardness. In addition, please also refer to FIG. 6. According to the embodiment as illustrated in FIG. 6, the second elastic layer 12 can be composed of a plurality of coiled elastic threads 16, and the elastic threads 16 are flexible and have elasticity. For instance, the elastic threads 16 can be made of plastic materials, such as TPU (Thermoplastic polyurethane), POE (Polyolefin Elastomer), PE (polyethylene), EVA (Ethylene vinyl acetate), PP (Polypropylene), PVC (Polyvinylchlorid), PET (polyethylene terephthalate) and other plastic materials, or a mixture of the aforementioned plastic materials. Similarly, for different design requirements, it is feasible to choose the elastic threads 16 with different thread diameters, to adjust its original spacing or curling between the elastic threads 16, or to use different materials and/or thicknesses of the elastic threads 16, etc., such that the elastic threads 16 can have different densities, and thus provide different supporting effects and hardness for the second elastic layer 12. In one embodiment of the present invention as shown in FIG. 6, for example, the second elastic layer 12 provides a second arc surface hardness in the region 60 which is adjacent to the second arc surface 132. The second elastic layer 12 provides a second bottom surface hardness in the region 61 which is adjacent to the second bottom surface 122. The region between the second arc surface 132 and the second bottom surface 122 of the second elastic layer 12 is a second intermediate layer 62, and the second elastic layer 12 provides a second intermediate layer hardness in the second intermediate layer 62. When a spacing between the elastic threads 16 disposed adjacent to the second arc surface 132 of the second elastic layer 12 is less than a spacing between the elastic threads 16 disposed adjacent to the second bottom surface 122 of the second elastic layer 12, and the spacing between the elastic threads 16 disposed adjacent to the second bottom surface 122 of the second elastic layer 12 is less than a spacing between the elastic threads 16 disposed in the second intermediate layer 62 of the second elastic layer 12, the present invention achieves in controlling the second arc surface hardness greater than the second bottom surface hardness, and the second bottom surface hardness greater than the second intermediate layer hardness. Besides, according to the embodiment of the present invention, these coiled elastic threads 15, 16 are also connected vertically to each other, and therefore providing longitudinal supporting forces. The first elastic layer 11 and the second elastic layer 12 are allowed to be made of different materials. According to the embodiment of the present invention, the first arc surface hardness of the first elastic layer 11 is greater than the first bottom surface hardness, and the first bottom surface hardness is greater than the first intermediate layer hardness. The second arc surface hardness of the second elastic layer 12 is greater than the second bottom surface hardness, and the second bottom surface hardness is greater than the second intermediate layer hardness. And, the first arc surface hardness, the first bottom surface hardness, the first intermediate layer hardness, the second arc surface hardness, the second bottom surface hardness, and the second intermediate layer hardness are different. As a result, by adjusting the spacing and distribution density of the elastic threads 15 in the first elastic layer 11 and the elastic threads 16 in the second elastic layer 12, the assistive device structure for positioning and pressure relief formed by the present invention is able to provide a variety (at least six different combination) of hardness, including the first arc surface hardness, the first bottom surface hardness, the first intermediate layer hardness, the second arc surface hardness, the second bottom surface hardness, and the second intermediate layer hardness. And based on such technical features, the present invention can be widely utilized to provide people with a variety of user experiences, thereby meeting different requirements and preferences of different users.

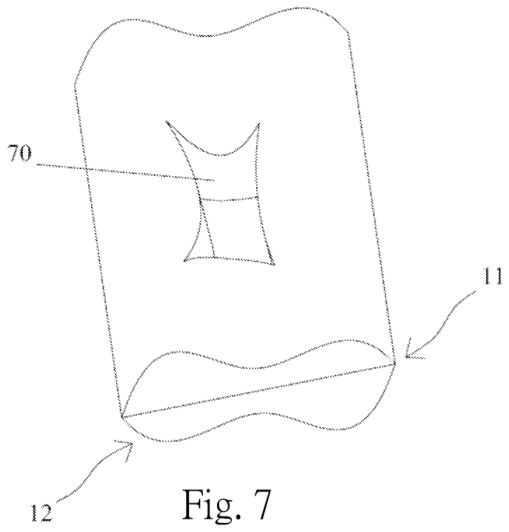

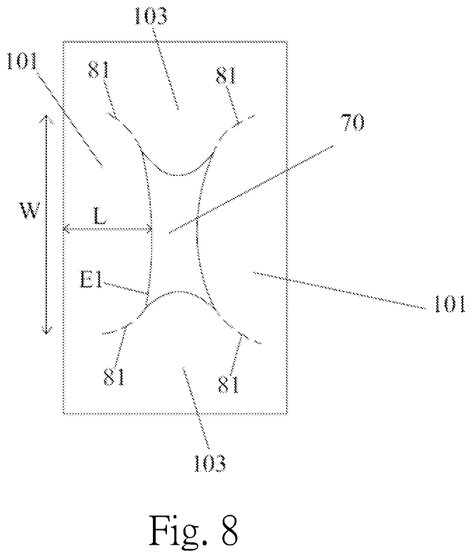

To be more specific, in order to comply with different users' needs and effectively reduce force concentration when the user is leaning against, the assistive device structure for positioning and pressure relief formed by the present invention may further include a hollow area which is disposed in either the first recess or the second recess. Please refer to FIG. 7 and FIG. 8 of the present invention. According to the assistive device structure for positioning and pressure relief disclosed by the present invention, the hollow area 70 can be further configured to reduce the force and pressure concentration when the user is leaning against it.

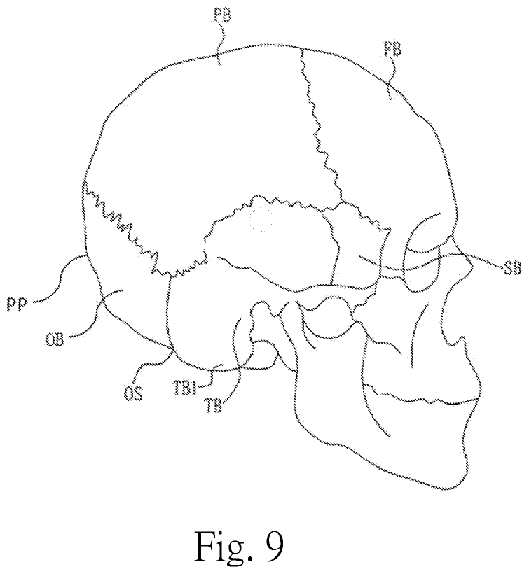

Subsequently, please refer to FIG. 8 for a detailed description, in which FIG. 8 shows an upper view of the embodiment of the present invention. As illustrated, each of the first protrusions and second protrusions disposed on opposite sides of the hollow area 70 includes a supporting portion 101, respectively. A width W of the supporting portion 101 is greater than a length L of the supporting portion 101. And, an edge E1 of the supporting portion 101 adjacent to the hollow area 70 is an arc. A peripheral portion 103 of the first elastic layer and the second elastic layer is configured between the adjacent supporting portions 101. Please refer to FIG. 9 for symbolic elements at the same time. As illustrated, since a human skull is composed of multiple bones, including FB (Frontal bone), SB (Sphenoid bone), PB (Parietal bone), OB(Occipital bone), TB, TB1 (Temporal bone), OS (Occipitomastoid Suture), PP (Posterior Pole of Skull, Occipital Bone) and so on, when the user is leaning against the disclosed assistive device structure for positioning and pressure relief, the supporting portion 101 disposed on one side of the hollow area 70 is used to support the occipital bone and cervical spine of the user, which correspond to the occipital bone OB as labeled in the schematic skull diagram in FIG. 9. As for another supporting portion 101 disposed on opposite side of the hollow area 70, it is used to support the parietal bone of the user, which corresponds to the parietal bone PB as labeled in the schematic skull diagram in FIG. 9. The hollow area 70 itself is used to accommodate the posterior pole of occipital bone PP (there in between the parietal bone PB and the occipital bone OB) of the user. Therefore, it is believed that by employing the device structure proposed by the present invention, the cervical vertebrae of the user can be pulled apart by the two supporting portions 101 disposed on the different opposite sides, thereby providing various stiffness and comfort for the user. Meanwhile, different supporting effects, including supporting heights and supporting hardness, may also be provided due to different heights of the first protrusions in the first elastic layer and of the second protrusions in the second elastic layer.

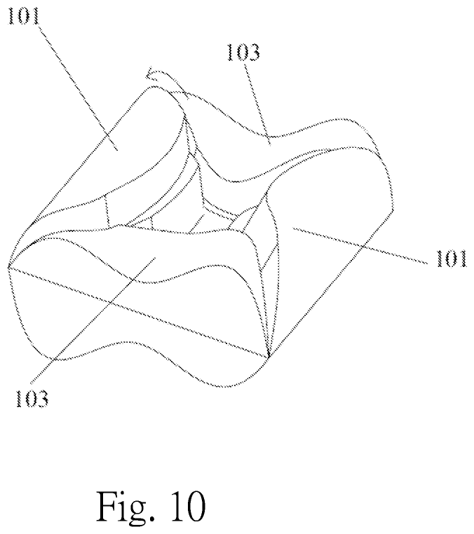

And furthermore, as shown in FIG. 8, according to the embodiment of the present invention, a plurality of cutting lines 81 are formed along the hollow area 70 in an extending direction of the edge E1 of the supporting portion 101. And, each cutting line 81 goes deep into one of the supporting portions 101, such that the supporting portions 101 are movable relative to the peripheral portions 103, as shown in FIG. 10. In one embodiment of the present invention, a length of each cutting line 81, for example, can be designed as 2 to 4 cm. Therefore, when the user is leaning against the disclosed assistive device structure for positioning and pressure relief in the present invention, the height of the peripheral portion 103 will be higher than the height of the supporting portion 101, which can effectively prevent the user's head from moving and avoid a crick in the user's neck.

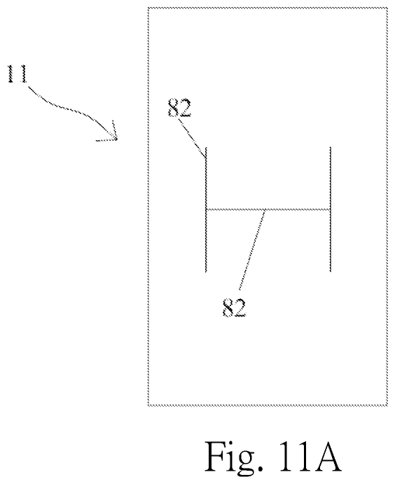

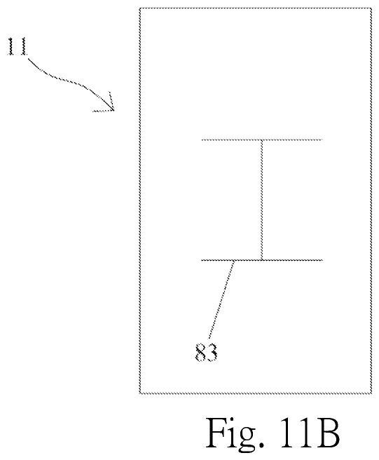

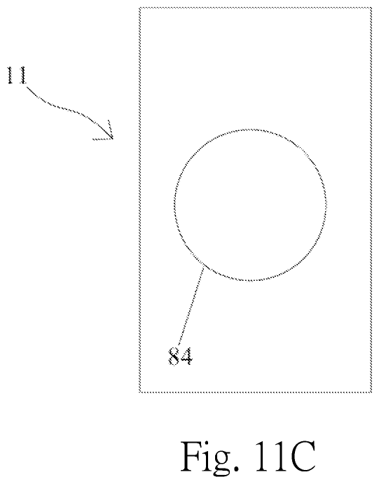

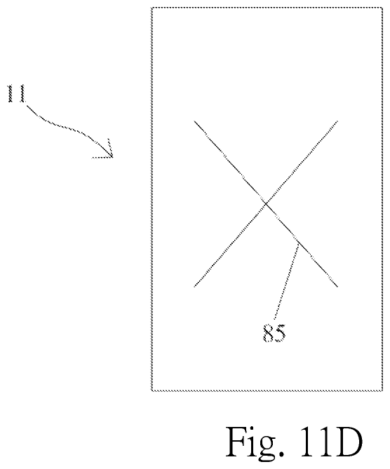

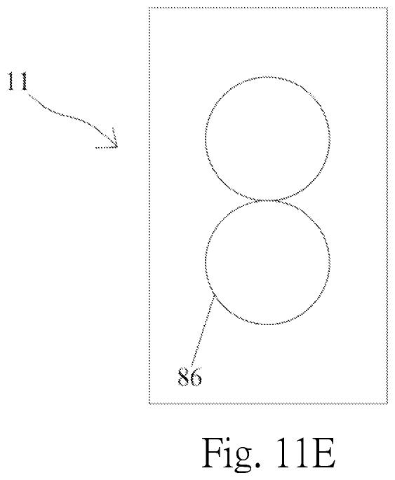

And moreover, the abovementioned cutting lines are not limited to the patterns as disclosed in the embodiment of FIG. 8. Please refer to FIG. 11A, FIG. 11B, FIG. 11C, FIG. 11D, and FIG. 11E of the present invention for several more different embodiments. Among these alternative embodiments of the present invention, the plurality of cutting lines configured in the first recess or in the second recess may also be formed in a symmetrical pattern, including the H-shaped cutting line 82 as shown in FIG. 11A, the I-shaped cutting line 83 as shown in FIG. 11B, the O-shaped cutting line 84 as shown in FIG. 11C, the X-shaped cutting line 85 as shown in FIG. 11D, and the figure-eight “8”-shaped cutting line 86 as shown in FIG. 11E. These symmetrical patterns provide a plurality of various embodiments of the present invention as well and still fall within the scope of the invention and its equivalent.

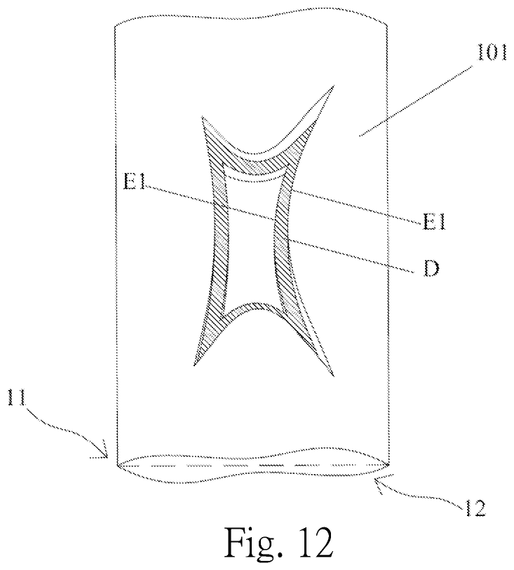

In addition, please refer to FIG. 12, in which the first protrusion of the first elastic layer 11 having a higher height is disposed in a same axial direction with the second protrusion of the second elastic layer 12 having a lower height, and the first protrusion of the first elastic layer 11 having a lower height is disposed in another axial direction with the second protrusion of the second elastic layer 12 having a higher height. According to such embodiment as shown in FIG. 12, a gap D will be formed between the edge E1 of the supporting portion 101 in the first elastic layer 11 and the edge E1 of the supporting portion 101 in the second elastic layer 12. Such a gap D is provided to prevent the supporting portion 101 which supports the occipital bone, from over bending or deformation when the user is leaning against the disclosed assistive device structure for positioning and pressure relief.

On the other hand, the present invention can be further verified by adopting some easy and commonly used testing methods, and it has thus been confirmed that the disclosed device structure of the present invention is able to provide not only multi-axial pressure relief, but also superior integrity, consistency, invariance, fatigue resistance, and acceptable reliability. Among them, the testing methods may include, for example, using heart rate variability (HRV) analysis, autonomic nervous systems, brain waves, sleep detection or personal portable health management detection instruments, and so on to verify the treatment principle together with the applied device structure. According to one embodiment of the present invention, when it is applied to sleep detection, then a sensor, for example, a sensor for measuring body position, can be further embedded in the first elastic layer 11 or in the second elastic layer 12 according to the practical application levels, such that by using the sensor, the user's sleep or leaning, using and other conditions can be detected and measured.

Therefore, to sum up, according to the foregoing several embodiments and technical contents disclosed in the present invention, those skilled in the art are able to make variations on their own when the actual implementation requirements must be considered, and yet those variations should still fall into the scope of the invention, since the several exemplary embodiments listed in the above foregoing paragraphs are merely provided for better understandings of the main technical features of the present invention, and not intended to limit the scope of the invention, so that those skilled in the art are able to understand and implement them accordingly.

As a result, based on the abovementioned embodiments provided by the present invention, it is obvious that the disclosed assistive device structure for positioning and pressure relief of the invention is a certain kind of aid device that is adjustable, tunable, and personally physiological, which successfully replaces the conventional manual therapy. Moreover, the user's position can be further adjusted when heights, hardness, supporting levels, angles, and arcs of the device structure are further taken into consideration. And as such, it effectively helps the user to obtain non-linear arc stretching, traction, and relaxation. According to the disclosed assistive device structure for positioning and pressure relief of the invention, it can be applied not only in cervical vertebrae, thoracic vertebrae, and lumbar vertebrae extension, which usually include six levels of stretches and generate different tensions according to different bending moments, but also in spine extension. For instance, since a human spine is about 60 cm, 10 cm can be taken as a spacing starting from the sacral vertebra to the occipital bone to provide six different curvatures of stretching. Besides, the curvature may also be adjusted for further providing arc stretching, turning stretching, lateral stretching, or pelvic stretching.

Therefore, by employing the device structure configuration of the present invention, the Applicant asserts that the present invention is instinct, effective, and highly competitive for incoming technologies, industries, and researches developed in the future. It is obvious that the technical features, means, and effects achieved by the present invention are significantly different from the current solutions and can not be accomplished easily by those who are familiar with the industry. As a result, it is believed that the present invention is indeed characterized by patentability and shall be patentable soon in a near future.

It will be apparent to those skilled in the art that various modifications and variations can be made to the present invention without departing from the scope or spirit of the invention. In view of the foregoing, it is intended that the present invention covers modifications and variations of this invention provided they fall within the scope of the invention and its equivalent.

What is claimed is:

1. An assistive device structure for positioning and pressure relief, comprising:
a first elastic layer, including a first bottom surface and a first arc surface, wherein the first bottom surface and the first arc surface are opposite, the first arc surface includes two first protrusions and a first recess which is formed between the two first protrusions, and the two first protrusions have different heights; and
a second elastic layer, including a second bottom surface and a second arc surface, wherein the second bottom surface and the second arc surface are opposite, the second arc surface includes two second protrusions and a second recess which is formed between the two second protrusions, the two second protrusions have different heights, and wherein the first bottom surface of the first elastic layer and the second bottom surface of the second elastic layer are attached, so as to form the assistive device structure for positioning and pressure relief for a user to lean against.

2. The assistive device structure for positioning and pressure relief of claim 1, wherein the first elastic layer and the second elastic layer are attached to each other by using a high-frequency encapsulation process, a sealing process, a bagging process, or a thermoforming process.

3. The assistive device structure for positioning and pressure relief of claim 1, wherein the first elastic layer and the second elastic layer are attached to each other by using an integrally molding process.

4. The assistive device structure for positioning and pressure relief of claim 1, wherein when the first elastic layer and the second elastic layer are attached to each other, one of the two first protrusions with a higher height in the first elastic layer and one of the two second protrusions with a higher height in the second elastic layer are commonly disposed in a same axial direction, and another of the two first protrusions with a lower height in the first elastic layer and another of the two second protrusions with a lower height in the second elastic layer are commonly disposed in another axial direction.

5. The assistive device structure for positioning and pressure relief of claim 1, wherein when the first elastic layer and the second elastic layer are attached to each other, one of the two first protrusions with a higher height in the first elastic layer and one of the two second protrusions with a lower height in the second elastic layer are commonly disposed in a same axial direction, and another of the two first protrusions with a lower height in the first elastic layer and another of the two second protrusions with a higher height in the second elastic layer are commonly disposed in another axial direction.

6. The assistive device structure for positioning and pressure relief of claim 1, wherein each of the first elastic layer and the second elastic layer is composed of a plurality of coiled elastic threads, and the elastic threads are made of plastic materials

7. The assistive device structure for positioning and pressure relief of claim 6, wherein the first elastic layer provides a first arc surface hardness in a region which is adjacent to the first arc surface, the first elastic layer provides a first bottom surface hardness in a region which is adjacent to the first bottom surface, the first elastic layer provides a first intermediate layer hardness in a region which is between the first arc surface and the first bottom surface, and wherein the second elastic layer provides a second arc surface hardness in a region which is adjacent to the second arc surface, the second elastic layer provides a second bottom surface hardness in a region which is adjacent to the second bottom surface, the second elastic layer provides a second intermediate layer hardness in a region which is between the second arc surface and the second bottom surface, and wherein the first arc surface hardness, the first bottom surface hardness, the first intermediate layer hardness, the second arc surface hardness, the second bottom surface hardness, and the second intermediate layer hardness are different, and wherein the first arc surface hardness is greater than the first bottom surface hardness, the first bottom surface hardness is greater than the first intermediate layer hardness, the second arc surface hardness is greater than the second bottom surface hardness, and the second bottom surface hardness is greater than the second intermediate layer hardness

8. The assistive device structure for positioning and pressure relief of claim 7, wherein the region which is between the first arc surface and the first bottom surface of the first elastic layer is a first intermediate layer, a spacing between the elastic threads disposed adjacent to the first arc surface of the first elastic layer is less than a spacing between the elastic threads disposed adjacent to the first bottom surface of the first elastic layer, and the spacing between the elastic threads disposed adjacent to the first bottom surface of the first elastic layer is less than a spacing between the elastic threads disposed in the first intermediate layer of the first elastic layer, such that the first arc surface hardness is greater than the first bottom surface hardness, and the first bottom surface hardness is greater than the first intermediate layer hardness.

9. The assistive device structure for positioning and pressure relief of claim 7, wherein the region which is between the second arc surface and the second bottom surface of the second elastic layer is a second intermediate layer, a spacing between the elastic threads disposed adjacent to the second arc surface of the second elastic layer is less than a spacing between the elastic threads disposed adjacent to the second bottom surface of the second elastic layer, and the spacing between the elastic threads disposed adjacent to the second bottom surface of the second elastic layer is less than a spacing between the elastic threads disposed in the second intermediate layer of the second elastic layer, such that the second arc surface hardness is greater than the second bottom surface hardness, and the second bottom surface hardness is greater than the second intermediate layer hardness.

10. The assistive device structure for positioning and pressure relief of claim 1, further comprising a hollow area which is disposed in either the first recess or the second recess to reduce force and pressure concentration when the user is leaning against.

11. The assistive device structure for positioning and pressure relief of claim 10, wherein each of the two first protrusions and the two second protrusions disposed on opposite sides of the hollow area respectively includes a supporting portion, a peripheral portion of the first elastic layer and the second elastic layer is configured between the adjacent supporting portions, and wherein when the user is leaning against, the supporting portion disposed on one side of the hollow area is used to support occipital bone and cervical spine of the user, the supporting portion disposed on opposite side of the hollow area is used to support parietal bone of the user, and the hollow area is used to accommodate posterior pole of occipital bone of the user, such that various supporting heights and supporting hardness are provided due to the different heights of the two first protrusions in the first elastic layer and of the two second protrusions in the second elastic layer.

12. The assistive device structure for positioning and pressure relief of claim 11, wherein a width of the supporting portion is greater than a length of the supporting portion.

13. The assistive device structure for positioning and pressure relief of claim 11, wherein an edge of the supporting portion adjacent to the hollow area is an arc.

14. The assistive device structure for positioning and pressure relief of claim 13, wherein a plurality of cutting lines are formed along the hollow area in an extending direction of the edge of the supporting portion, each of the plurality of cutting lines goes deep into the supporting portion, such that the supporting portion is movable relative to the peripheral portion.

15. The assistive device structure for positioning and pressure relief of claim 14, wherein a length of each of the plurality of cutting lines is 2 to 4 cm.

16. The assistive device structure for positioning and pressure relief of claim 14, wherein a height of the peripheral portion is higher than that of the supporting portion when the user is leaning against it so as to prevent the user's head from moving and avoid a crick in the user's neck.

17. The assistive device structure for positioning and pressure relief of claim 1, wherein a plurality of cutting lines are configured in the first recess or in the second recess, and the plurality of cutting lines are formed in a symmetrical pattern.

18. The assistive device structure for positioning and pressure relief of claim 1, wherein a sensor is further embedded in the first elastic layer or in the second elastic layer for detecting and measuring the sleep or leaning conditions of the user.

19. The assistive device structure for positioning and pressure relief of claim 11, wherein when the first elastic layer and the second elastic layer are attached to each other, one of the two first protrusions with a higher height in the first elastic layer and one of the two second protrusions with a lower height in the second elastic layer are commonly disposed in a same axial direction, and another of the two first protrusions with a lower height in the first elastic layer and another of the two second protrusions with a higher height in the second elastic layer are commonly disposed in another axial direction, a gap is formed between an edge of the supporting portion in the first elastic layer and an edge of the supporting portion in the second elastic layer.
